(19) United States
(12) Patent Application Publication
(10) Pub. No.: US 2026/0199377 A1
(43) Pub. Date: Jul. 16, 2026

(54) CANNABINOIDS FOR TREATING ESSENTIAL TREMOR IN PATIENTS

(71) Applicant: Jazz Pharmaceuticals Research UK Limited, Sittingbourne, Kent (GB)

(72) Inventors: Andrew C. MCCREARY, Kent (GB); Sally LOOMIS, Kent (GB); David VIRLEY, Kent (GB)

(21) Appl. No.: 19/134,833

(22) PCT Filed: Dec. 1, 2023
(86) PCT No.: PCT/EP2023/084007

§ 371 Date: Jun. 2, 2025

Related U.S. Application Data
(60) Provisional application No. 63/385,937, filed on Dec. 2, 2022.

(51) Int. Cl. A61K 31/00; A61K 31/015; A61K 31/045; A61K 31/355; A61K 31/575; A61P 25/14

(52) U.S. Cl. CPC A61K 31/658; A61K 31/015; A61K 31/045; A61K 31/355; A61K 31/575; A61P 25/14

(57) ABSTRACT

The present disclosure provides methods for treating a tremor in a subject In one aspect, the method comprises administering to the subject a therapeutically effective amount of a pharmaceutical composition comprising Δ-9-tetrahydrocannabinol (THC) and cannabidiol (CBD), wherein the THC and CBD are present at a weight ratio ranging from about 0.5:1.5 to about 1.5:0.5.

### CROSS-REFERENCE TO RELATED APPLICATIONS

This application claims benefit of U.S. Provisional Application No. 63/385,937, filed Dec. 2, 2022, the contents of which is incorporated herein by reference in its entirety.

### BACKGROUND OF THE INVENTION

Tremor is involuntary and rhythmic muscle contraction and relaxation in a subject resulting in movement (e.g., oscillation, shaking, twitching, trembling, twisting, etc.) of one or more body parts of the subject. As such, tremor disrupts voluntary bodily movement. This disruption can be severe and can affect the ability to perform basic tasks such as eating and drinking.

There are numerous forms (types) and sources of tremor. Essential tremor (“ET”), a neurological disorder (also referred to as “ET disorder”), is the most common form, usually affecting the hands and arms but also affecting the head, neck, legs, and other body parts. ET can be distinguished from tremor that results from other disorders or known causes, such as Parkinson's disease or head trauma. In ET, tremor can occur during movement (action-related tremor) and can be less noticeable with rest. Usually, ET is progressive. No cure for ET has been found.

Propranolol hydrochloride (“propranolol”) is the only FDA-approved drug for ET. Propranolol can reduce tremor amplitude. However, propranolol is not known to reduce tremor frequency. Adverse reactions observed in patients using propranolol include cardiovascular, central nervous system, gastrointestinal, allergic, respiratory, hematologic, autoimmune, epidermal, mucosal, and genitourinary events.

Primidone has also been used off-label for treating ET. However, a major drawback of primidone is the possibility of acute toxic reaction resulting in nausea, vomiting, ataxia, sedation, and giddiness. This adverse reaction can present at therapy initiation, and even when the dose is slowly titrated.

There is a need for new therapies for treating tremor, including ET.

### SUMMARY OF THE INVENTION

The present disclosure provides methods of treating tremor in subjects. In embodiments, the present disclosure provides a method of treating a tremor in a subject, the method comprising administering to the subject a therapeutically effective amount of a pharmaceutical composition comprising Δ-9-tetrahydrocannabinol (THC) and cannabidiol (CBD), wherein the THC and CBD are present at a weight ratio ranging from about 0.5:1.5 to about 1.5:0.5 THC to CBD. In embodiments, the subject is a human. In embodiments, the tremor comprises essential tremor. In embodiments, the administration reduces an intensity of the tremor compared to a control. In embodiments, the administration reduces an amplitude of the tremor compared to a control. In embodiments, the administration reduces an integral of the amplitude of the tremor compared to a control. In embodiments, the administration reduces a periodicity of the tremor compared to a control. In embodiments, about 0.001 to about 1,000 mg/kg/day THC and about 0.001 to about 1,000 mg/kg/day CBD are administered. In embodiments, about 0.001 to about 300 mg/kg/day THC and about 0.001 to about 300 mg/kg/day CBD are administered. In embodiments, about 0.1 to about 100 mg/kg/day THC and about 0.1 to about 100 mg/kg/day CBD are administered. In embodiments, about 0.1 to about 100 mg THC and about 0.1 to about 100 mg CBD are administered. In embodiments, the THC and CBD are present in a weight ratio ranging from about 0.9:1.1 to about 1.1:0.9. In embodiments, the THC and CBD are present in a weight ratio of about 1.1:0.9. In embodiments, the THC and CBD are present in a weight ratio of about 1.1:1. In embodiments, the pharmaceutical composition comprises a botanical drug substance comprising THC and CBD. In embodiments, the pharmaceutical composition further comprises one or more cannabinoids in addition to THC and CBD. In embodiments, the one or more cannabinoids comprise cannabidiolic acid (CBDA), cannabidivarin (CBDV), cannabidiol-C1 (CBD-C1), cannabidiol-C4 (CBD-C4), tetrahydrocannabivarin (THCV), cannabigerol (CBG), hydroxy cannabidiol (OH-CBD), butyl-cannabidiol (CBD-C4), cannabicyclol (CBL), or a combination thereof. In embodiments, the pharmaceutical composition further comprises one or more terpenes. In embodiments, the pharmaceutical composition further comprises one or more sesquiterpenes. In embodiments, the one or more terpenes or sesquiterpenes comprise beta-farnesene, selina-3,7(11)-diene, guaia-3,9-diene, trans-caryophyllene, alpha-caryophyllene, trans-nerolidol, myrcene, trans-phytol, squalene, alpha-tocopherol, or a combination thereof. In embodiments, the pharmaceutical composition further comprises one or more sterols. In embodiments, the one or more sterols comprise beta-sitosterol, beta-amyrin, campesterol, lupeol, or a combination thereof. In embodiments, the pharmaceutical composition is administered orally. In embodiments, the pharmaceutical composition is a spray. In embodiments, the pharmaceutical composition is a tablet, a gel capsule, a mucoadhesive gel, a powder, a granule, or an extrudate.

### BRIEF DESCRIPTION OF THE DRAWINGS

FIGS. 1A-C provide plots of observational scores (“OS”) for subjects 20 minutes (FIG. 1A), 40 minutes (FIG. 1B), and 60 minutes (FIG. 1C) after being administered vehicle (saline; i.p. at 5 mL/kg) or harmaline (i.p. at 5 mL/kg) after acute administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). OS of 0, 1, 2, 3, and 4 correspond to “No tremor”, “Occasional tremor affecting only head and neck”, “Intermittent tremor affecting whole body”, “Persistent tremor affecting whole body and tail”, and “Severe tremor rendering subject unable to stand”, respectively. Data were analyzed with a Kruskal-Wallis followed by Dunn's multiple comparison test (GraphPad, Prism v9.0.0). ##, ###, and #### indicate p<0.01, 0.001, and 0.0001, respectively, when compared to the vehicle/harmaline group at the same timepoint. Boxplot graphs show median±IQR with min/max shown as whiskers.

FIGS. 2A-C provide plots of average amplitude (dBV) as a function of frequency for subjects 20 minutes (FIG. 2A), 40 minutes (FIG. 2B), and 60 minutes (FIG. 3C) after being administered vehicle (saline; i.p. at 5 mL/kg) or harmaline (i.p. at 5 mL/kg) after acute administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline), as measured for 5 minutes using the Tremor Monitor described herein and calculated using a Fast Fourier transformation. SEM is shown as whiskers.

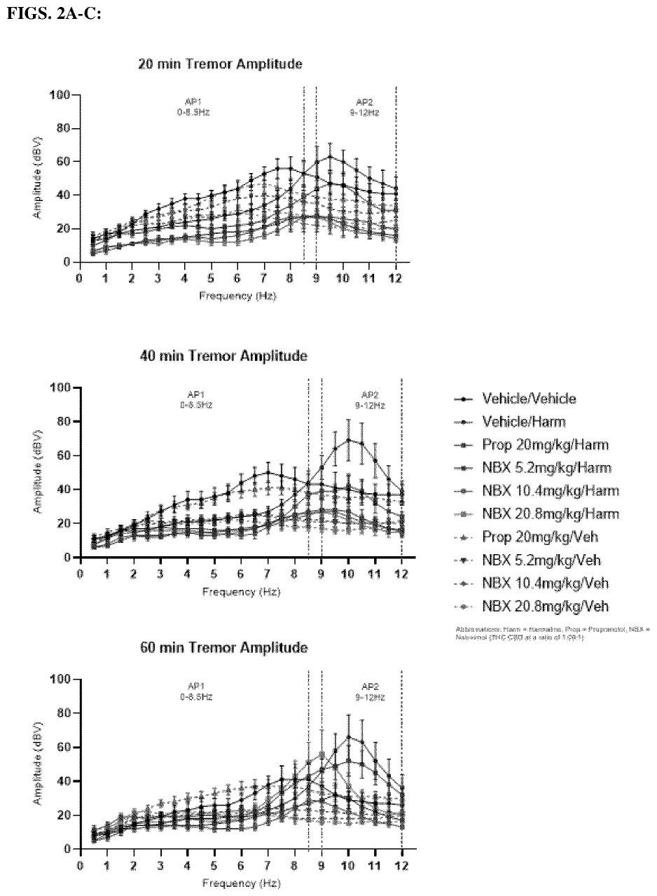

FIGS. 3A-C provide plots of Motion Power Percentage (“MPP”) as defined herein for subjects 20 minutes (FIG. 2A), 40 minutes (FIG. 2B), and 60 minutes (FIG. 3C) after being administered vehicle (saline; i.p. at 5 mL/kg) or harmaline (i.p. at 5 mL/kg) after acute administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). Data were expressed in mean±SEM and were analyzed with a One-Way ANOVA followed by Dunnett's multiple comparison analysis. *, ***, and **** indicate p<0.05, 0.001, and 0.0001, respectively, when compared to the vehicle/vehicle group at the same timepoint. #, ##, ###, and #### indicate p<0.05, 0.01, 0.001, and 0.0001, respectively, when compared to the vehicle/harmaline group at the same timepoint.

FIGS. 4A-C provide plots of Tremor Index (“TI”) as defined herein for subjects 20 minutes (FIG. 2A), 40 minutes (FIG. 2B), and 60 minutes (FIG. 3C) after being administered vehicle (saline; i.p. at 5 mL/kg) or harmaline (i.p. at 5 mL/kg) after acute administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). Data were expressed in mean±SEM and were analyzed with a One-Way ANOVA followed by Dunnett's multiple comparison analysis. *** and **** indicate p<0.001 and 0.0001, respectively, when compared to the vehicle/vehicle group at the same timepoint. #, ##, ###, and #### indicate p<0.05, 0.01, 0.001, and 0.0001, respectively, when compared to the vehicle/harmaline group at the same timepoint.

FIGS. 5A-C provide plots of total number of tremor events (FIG. 5A), total average length of one tremor event (FIG. 5B), and total cumulative time of tremor events (FIG. 5C) for subjects 20, 40, and 60 minutes after being administered vehicle (saline; i.p. at 5 mL/kg) or harmaline (i.p. at 5 mL/kg) after acute administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). Data were analyzed with a Kruskal-Wallis followed by Dunn's multiple comparison test (GraphPad, Prism v9.0.0). *** and **** indicate p<0.001 and 0.0001, respectively, when compared to the vehicle/vehicle group at the same timepoint. #, ##, ###, and #### indicate p<0.05, 0.01, 0.001, and 0.0001, respectively, when compared to the vehicle/harmaline group at the same timepoint.

FIGS. 6A-C provide plots of observational scores (“OS”) for subjects 20 minutes (FIG. 6A), 40 minutes (FIG. 6B), and 60 minutes (FIG. 6C) after being administered vehicle (saline; i.p. at 5 mL/kg) or harmaline (i.p. at 5 mL/kg) after the final administration of once daily administration for 10 days (“sub-chronic administration”) of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). OS of 0, 1, 2, 3, and 4 correspond to “No tremor”, “Occasional tremor affecting only head and neck”, “Intermittent tremor affecting whole body”, “Persistent tremor affecting whole body and tail”, and “Severe tremor rendering subject unable to stand”, respectively. Data were analyzed with a Kruskal-Wallis followed by Dunn's multiple comparison test (GraphPad, Prism v9.0.0). ##, ###, and #### indicate p<0.01, 0.001, and 0.0001, respectively, when compared to the vehicle/harmaline group at the same timepoint. Boxplot graphs show median±IQR with min/max shown as whiskers.

FIGS. 7A-C provide plots of average amplitude (dBV) as a function of frequency for subjects 20 minutes (FIG. 7A), 40 minutes (FIG. 7B), and 60 minutes (FIG. 7C) after being administered vehicle (saline; i.p. at 5 mL/kg) or harmaline (i.p. at 5 mL/kg) after the final administration of sub-chronic administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline), as measured for 5 minutes using the Tremor Monitor described herein and calculated using a Fast Fourier transformation. SEM is shown as whiskers.

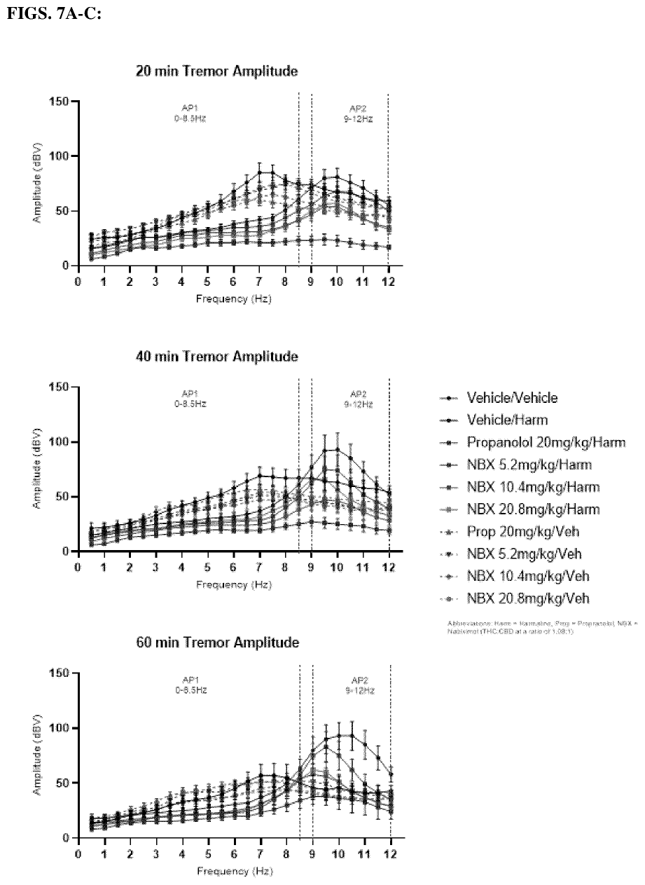

FIGS. 8A-C provide plots of Motion Power Percentage (“MPP”) as defined herein for subjects 20 minutes (FIG. 8A), 40 minutes (FIG. 8B), and 60 minutes (FIG. 8C) after being administered vehicle (saline; i.p. at 5 mL/kg) or harmaline (i.p. at 5 mL/kg) after the final administration of sub-chronic administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). Data were expressed in mean±SEM and were analyzed with a One-Way ANOVA followed by a Tukey's multiple comparison analysis. *, ***, and **** indicate p<0.05, 0.001, and 0.0001, respectively, when compared to the vehicle/vehicle group at the same timepoint. #, ##, ###, and #### indicate p<0.05, 0.01, 0.001, and 0.0001, respectively, when compared to the vehicle/harmaline group at the same timepoint.

FIGS. 9A-C provide plots of Tremor Index (“TI”) as defined herein for subjects 20 minutes (FIG. 9A), 40 minutes (FIG. 9B), and 60 minutes (FIG. 9C) after being administered vehicle (saline; i.p. at 5 mL/kg) or harmaline (i.p. at 5 mL/kg) after the final administration of sub-chronic administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). Data were expressed in mean±SEM and were analyzed with a One-Way ANOVA followed by a Tukey's multiple comparison analysis. *** and **** indicate p<0.001 and 0.0001, respectively, when compared to the vehicle/vehicle group at the same timepoint. #, ##, ###, and #### indicate p<0.05, 0.01, 0.001, and 0.0001, respectively, when compared to the vehicle/harmaline group at the same timepoint.

FIGS. 10A-C provide plots of total number of tremor events (FIG. 10A), total average length of one tremor event (FIG. 10B), and total cumulative time of tremor events (FIG. 10C) for subjects 20, 40, and 60 minutes after being administered vehicle (saline; i.p. at 5 mL/kg) or harmaline (i.p. at 5 mL/kg) after the final administration of sub-chronic administration of vehicle (sesame oil; p.o. at 2 mL/kg 100 minutes before the harmaline or saline), propranolol (i.p. at 5 mL/kg 30 minutes before the harmaline or saline), or the pharmaceutical composition described herein (p.o. at 2 mL/kg 100 minutes before the harmaline or saline). Data were analyzed with a Kruskal-Wallis followed by a Tukey's multiple comparison test (GraphPad, Prism v9.0.0). and *** indicate p<0.001 and 0.0001, respectively, when compared to the vehicle/vehicle group at the same timepoint. #, ##, ###, and #### indicate p<0.05, 0.01, 0.001, and 0.0001, respectively, when compared to the vehicle/harmaline group at the same timepoint.

### DETAILED DESCRIPTION OF THE INVENTION

### Definitions

Certain terms are defined here. However, unless otherwise defined, all terms have the same meanings as commonly understood by a person of ordinary skill in the art to which this disclosure belongs.

Unless otherwise stated or otherwise evident by the context, every range herein refers to the range itself, all values therein, all ranges therebetween, and all combinations thereof. For example, a range of from about 1 to about 100 includes the range of from about 1 to about 100 itself, all values between about 1 and about 100 (e.g., 2, 3, 4, 5, 6, 7, 8, 9, 10, 15, 20, 25, 30, 35, 40, 45, 50, 55, 60, 65, 70, 75, 80, 85, 90, 95, and 99), all values therein, all ranges therebetween, and all combinations thereof.

The term “a” (or “an”) refers to one or more, or at least one, of that entity; for example, “a cannabinoid” refers to one or more cannabinoids, or at least one, cannabinoid. As such, the terms “a” (or “an”), “one or more,” and “at least one” are used interchangeably herein. Reference to “an element” by the indefinite article “a” (or “an”) does not exclude the possibility that more than one of the element is present, unless the context clearly requires that there is only one of the element.

The term “about” when immediately preceding a number or numerical range refers to an acceptable degree of variation in the art. In embodiments, “about” means plus or minus 10% of the referenced number(s) unless otherwise stated or otherwise evident by the context, and except where such a range would exceed 100% of a possible value, or be below 0% of a possible value, such as less than 0% of the content of an ingredient, or more than 100% of the total contents of a composition. When preceding a list or range of numbers, the term “about” applies to each number in the list or range. For example, “about 1, 2, or 3” means “about 1, about 2, or about 3” and “about 1 to 3” means “about 1 to about 3”.

The term “administering” refers to any route of administration, e.g., oral administration. Administering can also include prescribing a drug to be delivered to a subject, e.g., according to a particular dosing regimen, or filling a prescription for a drug that was prescribed to be delivered to a subject, e.g., according to a particular dosing regimen.

The term “botanical drug substance” refers to a drug substance (active agent or API) comprising plant materials.

The term “control” refers to a value of a characteristic (e.g., tremor intensity) of a subject at baseline (i.e., before the subject is administered the pharmaceutical composition disclosed herein) or an otherwise identical subject that is not administered the pharmaceutical composition disclosed herein. Similarly, “control group” refers to a group of subjects that serves as a basis for determining a value of a characteristic (e.g., tremor intensity) of the group at baseline (i.e., before the subjects of the group are administered the pharmaceutical composition disclosed herein) or an otherwise identical group that is not administered the pharmaceutical composition disclosed herein.

The term “effective amount” or “therapeutically effective amount” refers to an amount that can produce a therapeutic effect in a subject upon administration to the subject.

The term “tremor event” refers to involuntary and rhythmic muscle contraction and relaxation in a subject resulting in movement of one or more body parts of the subject for a duration. As such, tremor events in a subject are separated by durations during which involuntary and rhythmic muscle contraction and relaxation in the subject resulting in movement of one or more body parts of the subject does not occur.

The term “periodicity” refers to a number of tremor events in a subject during a length of time. As such, tremor resulting in more or fewer tremor events in a subject during a length of time has “higher” or “lower” periodicity, respectively.

The terms “oromucosal spray”, “buccal spray”, and “spray” are used interchangeably herein, and refer to a pharmaceutical formulation comprising cannabinoids that is administered by spraying into the oral mucosa, which may include, but is not limited to, the oral cavity and/or the pharynx.

The term “otherwise identical subject” refers to a subject whose characteristics are expected to be substantially the same as those of a subject that is administered the pharmaceutical composition disclosed herein. In embodiments, the otherwise identical subject is of substantially the same age, sex, and body weight as the subject that is administered the pharmaceutical composition disclosed herein. In embodiments, the otherwise identical subject has a substantially similar disease state as the subject that is administered the pharmaceutical composition disclosed herein.

The term “treating” means one or more of relieving, alleviating, delaying, reducing, reversing, improving, preventing, or managing at least one symptom of a condition, disease, or disorder in a subject. The term “treating” may also mean one or more of arresting, delaying the onset (i.e., the period prior to clinical manifestation of the condition), or reducing the risk of developing or worsening a condition, disease, or disorder in a subject. For example, treating may refer to reducing tremor intensity, frequency, periodicity, or amplitude.

### Tremor

The disclosure provides methods and pharmaceutical compositions that can be used to treat tremor. In embodiments, the tremor comprises essential tremor (ET) and/or enhanced physiologic tremor. In embodiments, the tremor comprises ET and enhanced physiologic tremor. In embodiments, the tremor comprises ET. In embodiments, the tremor comprises enhanced physiologic tremor.

In embodiments, the tremor comprises rest and/or action tremor. In embodiments, the tremor comprises action tremor. In embodiments, the action tremor comprises postural and/or kinetic tremor. In embodiments, the kinetic tremor comprises intention, task-specific, and/or isometric tremor.

In embodiments, tremor intensity can be measured by accelerometry, dynamics, electromyography, eyeblink conditioning, gyroscopy, piezoelectricity, medical imaging, scoring, and/or spiral analysis. Non-limiting examples of medical imaging include positron emission tomography and magnetic resonance imaging (e.g., fMRI). Non-limiting examples of scoring include the observational scoring scale described herein, The Essential Tremor Rating Assessment Scale (TETRAS), and the Fahn-Tolosa-Marin Tremor Rating Scale (FTMTRS). For example, tremor intensity can be measured by measuring: electrical activity of muscle tissue during one or more tremor events; tremor amplitude during one or more tremor events; and/or tremor periodicity. For example, tremor amplitude (“A”) can be measured as a function of frequency (“f”) (Hz) in terms of power (dBV) or linear or angular displacement (mm or degrees). As such, for example, tremor intensity can be measured by measuring A over a range of f (e.g., an integral or average of A, a derivative of A (e.g., the first, second, and/or third derivative), full width at half maximum of A, number of times that A exceeds a threshold value, etc.), or at a single f, for one or more tremor events. As such, for example, “reducing” (or “reduction of”) tremor intensity may refer to reducing, compared to a control or reference point, A at a single f for one tremor event, an average of A over a range of f for multiple tremor events in one or more subjects, or tremor periodicity in one or more subjects.

In embodiments, the tremor before treatment has a frequency or average frequency of about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25 or more Hz, one or more of any value therein or range therebetween, or combinations thereof, as measured during one or more tremor events. In embodiments, the subject is human and the tremor before treatment has a frequency or average frequency of about 4 to 12 Hz, as measured during one or more tremor events.

In embodiments, the tremor before treatment has an amplitude or average amplitude of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or more dBV, one or more of any value therein or range therebetween, or combinations thereof, as measured during one or more tremor events.

In embodiments, the tremor before treatment has an amplitude or average amplitude of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or more mm, one or more of any value therein or range therebetween, or combinations thereof, as measured during one or more tremor events.

In embodiments, the tremor before treatment has an observational score (“OS”) of 1, 2, 3, and/or 4 on the observational scoring scale described herein (and summarized in TABLE A below), as measured during one or more tremor events.

In embodiments, the tremor before treatment has a Tremor Index (“TI”) or average TI of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more dBV, one or more of any value therein or range therebetween, or combinations thereof, as measured during one or more tremor events.

In embodiments, the tremor before treatment has a Motion Power Percentage (“MPP”) or average MPP of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, one or more of any value therein or range therebetween, or combinations thereof, as measured during one or more tremor events. In embodiments, the tremor before treatment has an MPP or average MPP of about 40 to 80%, one or more of any value or range therein, or combinations thereof, as measured during one or more tremor events.

In embodiments, the tremor before treatment results in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more tremor events during a 5 minute period.

In embodiments, the tremor before treatment results in tremor events having an average length of about 1, 2, 3, 4, 5, 6, 7, 8, 9, or 10 or more seconds, or any value therein.

In embodiments, the tremor before treatment results in a subject spending about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 seconds in tremor during a 5 minute period.

In embodiments, the tremor before treatment results in a subject spending about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of a length of time in tremor.

In embodiments, the tremor before treatment corresponds to a TETRAS score of 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, or 64.

### Pharmaceutical Composition

The pharmaceutical composition described herein comprises cannabidiol (CBD) and tetrahydrocannabinol (THC). In embodiments, THC is present as the trans isomer, the cis isomer, or a combination thereof. In embodiments, the pharmaceutical composition further comprises one or more additional cannabinoids, including, but not limited to cannabidiolic acid (CBDA), cannabidivarin (CBDV), cannabidiol-C1 (CBD-C1), cannabidiol-C4 (CBD-C4), tetrahydrocannabivarin (THCV), cannabigerol (CBG), hydroxy cannabidiol (OH-CBD), butyl-cannabidiol (CBD-C4), cannabicyclol (CBL), or a combination thereof. In embodiments, the pharmaceutical composition further comprises one or more terpenes. In embodiments, the terpenes comprise one or more sesquiterpenes. Non-limiting examples of terpenes and sesquiterpenes include, but are not limited to, beta-farnesene, selina-3,7(11)-diene, guaia-3,9-diene, trans-caryophyllene, alpha-caryophyllene, trans-nerolidol, myrcene, trans-phytol, squalene, alpha-tocopherol, or a combination thereof. In embodiments, the pharmaceutical composition further comprises one or more sterols, including but not limited to beta-sitosterol, beta-amyrin, campesterol, lupeol, or a combination thereof. Table 1 below provides the structure of certain cannabinoids, terpenes, and sterols along with their standard abbreviations. The table below is not exhaustive and merely details the cannabinoids and other potential components of the pharmaceutical composition which are identified in the present application for reference.

TABLE 1
Non-limiting Examples of Components of the Pharmaceutical Composition and
Their Abbreviations

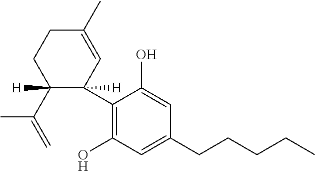 [SMILES: C=C(C)[C@@H]1CCC(C)=C[C@H]1c1c(O)cc(CCCCC)cc1O]

Cannabidiol (CBD)

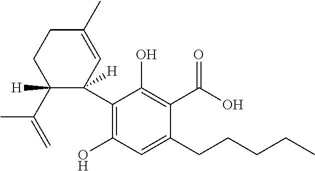 [SMILES: C=C(C)[C@@H]1CCC(C)=C[C@H]1c1c(O)cc(CCCCC)c(C(=O)O)c1O]

Cannabidiolic acid (CBDA)

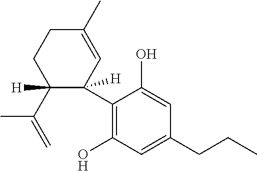 [SMILES: C=C(C)[C@@H]1CCC(C)=C[C@H]1c1c(O)cc(CCC)cc1O]

Cannabidivarin (CBDV)

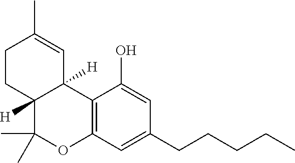 [SMILES: CCCCCc1cc(O)c2c(c1)OC(C)(C)[C@@H]1CCC(C)=C[C@@H]21]

trans-Tetrahydrocannabinol (THC)

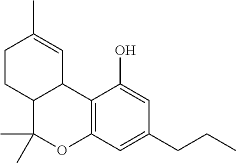 [SMILES: CCCc1cc(O)c2c(c1)OC(C)(C)C1CCC(C)=CC21]

Tetrahydrocannabivarin (THCV)

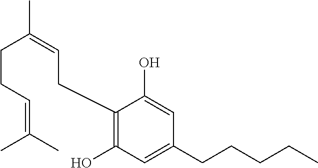 [SMILES: CCCCCc1cc(O)c(C/C=C(/C)CCC=C(C)C)c(O)c1]

Cannabigerol (CBG)

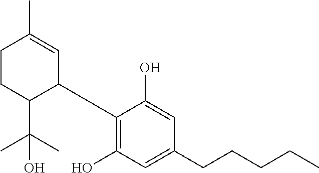 [SMILES: CCCCCc1cc(O)c(C2C=C(C)CCC2C(C)(C)O)c(O)c1]

OH-CBD (hydroxy cannabidiol)

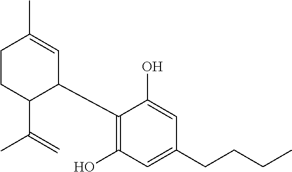 [SMILES: C=C(C)C1CCC(C)=CC1c1c(O)cc(CCCC)cc1O]

Butyl-cannabidiol (CBD-C4)

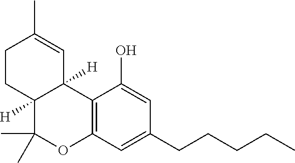 [SMILES: CCCCCc1cc(O)c2c(c1)OC(C)(C)[C@H]1CCC(C)=C[C@@H]21]

cis-THC (cis-Tetrahydrocannabinol)

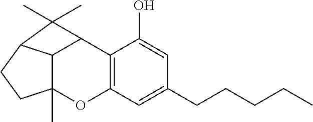 [SMILES: CCCCCc1cc(O)c2c(c1)OC1(C)CCC3C1C2C3(C)C]

CBL (Cannabicyclol)

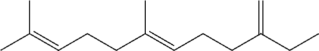

Beta-Farnesene

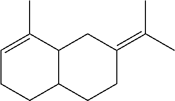

Selina-3,7(11)-diene

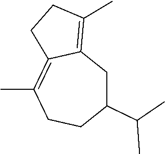

Guaia-3,9-diene

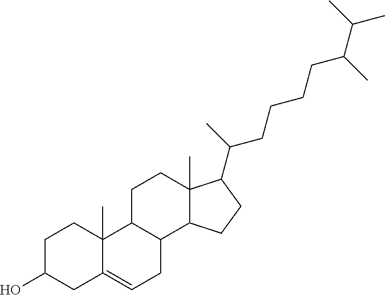 [SMILES: CC(C)C(C)CCCCC(C)C1CCC2C3CC=C4CC(O)CCC4(C)C3CCC12C]

Campesterol

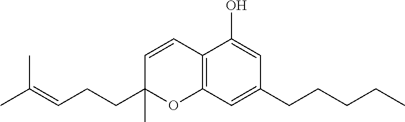 [SMILES: CCCCCc1cc(O)c2c(c1)OC(C)(CCC=C(C)C)C=C2]

Cannabichromene (CBC)

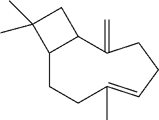

Trans-caryophyllene

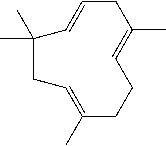

alpha-Caryophyllene

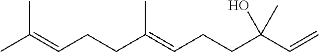 [SMILES: C=CC(C)(O)CC/C=C(\C)CCC=C(C)C]

Trans-nerolidol

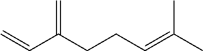

Myrcene

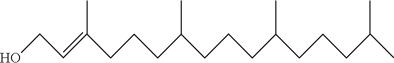 [SMILES: C/C(=C\CO)CCCC(C)CCCC(C)CCCC(C)C]

Trans-phytol

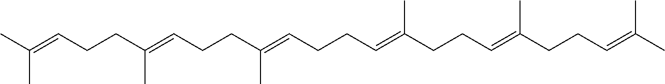 [SMILES: CC(C)=CCC/C(C)=C/CC/C(C)=C/CC/C=C(\C)CC/C=C(\C)CCC=C(C)C]

Squalene

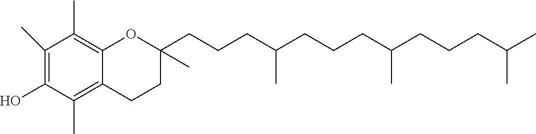 [SMILES: Cc1c(C)c2c(c(C)c1O)CCC(C)(CCCC(C)CCCC(C)CCCC(C)C)O2]

alpha-Tocopherol

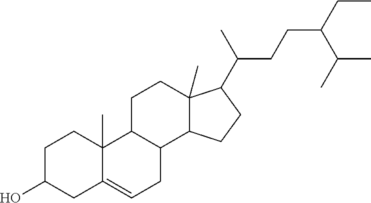 [SMILES: CCC(CCC(C)C1CCC2C3CC=C4CC(O)CCC4(C)C3CCC12C)C(C)C]

beta-Sitosterol

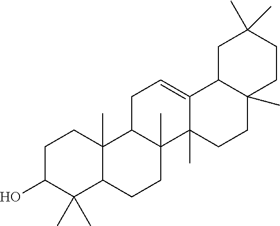

beta-Amyrin

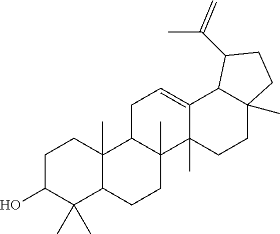

Lupeol

The cannabinoids, terpenes, and sterols described herein may be prepared synthetically or naturally from Cannabis. Cannabis is a genus of flowering plants in the family Cannabaceae, comprising the species Cannabis sativa, Cannabis indica, and Cannabis ruderalis. In embodiments, CBD and THC are prepared synthetically. In embodiments, both CBD and THC are extracted from Cannabis. When CBD and THC are extracted from plants, the drug substance may be referred to as a botanical drug substance. In embodiments, CBD and THC are present in the pharmaceutical composition as purified extracts. When CBD and THC are extracted and used in the pharmaceutical composition, CBD and THC may be the predominant cannabinoids in the pharmaceutical composition, but other cannabinoids or components (e.g., terpenes, sterols, etc.) may also exist in the pharmaceutical composition. Accordingly, in embodiments, the pharmaceutical composition described herein further comprises (in addition to CBD and THC), CBDA, CBDV, CBN, CBC, mono-methylated CBG (CBG MME), CBD-C1, CBD-C4, THCV, CBG, OH-CBD, CBL, DHC, various terpenes and sterols described herein (e.g., alpha-bergamotene, alpha-bisabolol, beta-farnesene, selina-3,7(11)-diene, guaia-3,9-diene, trans-caryophyllene, alpha-caryophyllene, trans-nerolidol, myrcene, trans-phytol, squalene, alpha-tocopherol, beta-sitosterol, beta-amyrin, campesterol, lupeol, or combinations thereof), or combinations thereof.

In embodiments, the pharmaceutical composition comprises CBD. In embodiments, CBD is extracted from a Cannabis. In embodiments, CBD is extracted from Cannabis and purified. In embodiments, the purified CBD extract comprises from 50% w/w to at least 99% w/w CBD based on the total weight of the extract. In embodiments, the CBD extract can comprise about 50% w/w, about 55% w/w, about 60% w/w, about 65% w/w, about 70% w/w, about 75% w/w, about 80% w/w, about 85% w/w, about 90% w/w, 95% w/w, about 96% w/w, about 97% w/w, about 98% w/w, about 99% w/w, or about 100% w/w CBD based on the total weight of cannabinoids in the extract, including all values and ranges therein. In embodiments, the CBD extract comprises at least 60% w/w CBD based on the total weight of the extract. In embodiments, the CBD extract comprises 98-99% w/w CBD based on the total weight of the extract. In embodiments, the CBD extract further comprises OH-CBD, CBDV, CBG, THC, CBC, CBG MME, Myrcene, Trans-Caryophyllene, Alpha-Caryophyllene, Trans-Nerolidol, Trans-Phytol, Squalene, Alpha-Tocopherol, Beta-Sitosterol, or Beta-Amyrin, or combinations thereof. In embodiments, the purified CBD extract may be combined with THC to form the pharmaceutical composition described herein. In embodiments, THC be prepared either synthetically or purified from Cannabis, and combined with the CBD. Methods for CBD synthesis and extraction are described in the following patent documents which are incorporated by reference in their entirety herein: U.S. Publication No. 2019/0231833 A1, (published Aug. 1, 2019), International Publication No. 2019/020738 (published Jan. 31, 2019), International Publication No. 2004/016277 A1 (published Feb. 26, 2004), U.S. Publication No. 2019/0160393 A1 (published May 30, 2019), and International Publication No. 2004/026802 (published Jan. 4, 2004).

In embodiments, the pharmaceutical composition comprises about 1 mg to about 1000 mg THC, e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 800, 905, 910, 915, 920, 925, 930, 935, 940, 945, 950, 955, 960, 965, 970, 975, 980, 985, 990, 995, or 1000 mg, including all values and ranges therein.

In embodiments, the pharmaceutical composition comprises about 1 mg to about 1000 mg CBD, e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 800, 905, 910, 915, 920, 925, 930, 935, 940, 945, 950, 955, 960, 965, 970, 975, 980, 985, 990, 995, or 1000 mg, including all values and ranges therein.

In embodiments, the pharmaceutical composition comprises a botanical drug substance, and the botanical drug substance comprises from about 10% w/w CBD to about 75% w/w CBD, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75% w/w CBD, including all values and ranges therein. In embodiments, the botanical drug substance in the pharmaceutical composition comprises from about 28% w/w CBD to about 35% w/w CBD. In embodiments, the botanical drug substance in the pharmaceutical composition comprises about 31.6% w/w CBD.

In embodiments, the pharmaceutical composition comprises a botanical drug substance, and the botanical drug substance comprises from about 10% w/w THC to about 75% w/w THC, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75% w/w THC, including all values and ranges therein. In embodiments, the botanical drug substance in the pharmaceutical composition comprises from about 32% w/w THC to about 39% w/w THC. In embodiments, the botanical drug substance in the pharmaceutical composition comprises about 35.6% w/w THC.

In embodiments, the pharmaceutical composition comprises about 1 mg to about 1000 mg THC and CBD in total, e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 800, 905, 910, 915, 920, 925, 930, 935, 940, 945, 950, 955, 960, 965, 970, 975, 980, 985, 990, 995, or 1000 mg, including all values and ranges therein. In embodiments, the pharmaceutical composition comprises about 5.2 mg THC and CBD in total. In embodiments, the pharmaceutical composition comprises about 10.4 mg THC and CBD in total. In embodiments, the pharmaceutical composition comprises about 20.8 mg THC and CBD in total.

In embodiments, the pharmaceutical composition comprises THC and CBD in a weight ratio ranging from about 0.9:1.1 to about 1.1:0.9, including all values and ranges therein. In embodiments, the pharmaceutical composition comprises THC and CBD in a weight ratio of about 1.1:0.9. In embodiments, the pharmaceutical composition comprises THC and CBD in a weight ratio of about 1.1:1.

In embodiments, the pharmaceutical composition comprises a THC extract. In embodiments, the THC extract comprises from 50% w/w to 100% w/w THC based on the total weight of the extract. In embodiments, the THC extract can comprise about 50% w/w, about 55% w/w, about 60% w/w, about 65% w/w, about 70% w/w, about 75% w/w, about 80% w/w, about 85% w/w, about 90% w/w, 95% w/w, about 96% w/w, about 97% w/w, about 98% w/w, about 99% w/w THC based on the total weight of the extract, including all values and ranges therein. In embodiments, the THC extract comprises at least 64% w/w THC based on the total weight of the extract. In embodiments, the THC extract comprises 98-99% w/w THC based on the total weight of cannabinoids in the extract. In some embodiments, the THC extract further comprises THCV, CBD, CBG, CBN, DHC, CBC, Myrcene, Trans-Caryophyllene, Alpha-Bergamotene, Beta-Farnesene, Alpha-Caryophyllene, Guaia-3,9-diene, Selina-3,7(11)-diene, Trans-Nerolidol, Alpha-Bisabolol, Trans-Phytol, Beta-Sitosterol, Beta-Amyrin, Alpha-Amyrin, or Lupeol, or combinations thereof. Methods for THC extraction are described in U.S. Publication No. 2005/0266108 (published Dec. 1, 2005) which is herein incorporated by reference in its entirety.

In embodiments, the pharmaceutical composition is orally deliverable. The terms “orally deliverable” or “oral administration” herein include any form of delivery of the pharmaceutical composition to a subject wherein the pharmaceutical composition contacts the mouth of the subject, whether or not the pharmaceutical composition is swallowed. Thus “oral administration” includes buccal and sublingual as well as esophageal administration.

In embodiments, the pharmaceutical composition is formulated as one or more dosage units. The terms “dose unit” and “dosage unit” herein refer to a portion of the pharmaceutical composition suitable for a single administration to provide a therapeutic effect. Such dosage units may be administered one to a plurality (i.e., 1 to about 10, 1 to 8, 1 to 6, 1 to 4 or 1 to 2) of times per day, or as many times as needed to elicit a therapeutic response.

In embodiments, the pharmaceutical composition further comprises a pharmaceutically acceptable carrier. In embodiments, the pharmaceutically acceptable carrier aids formulation of the pharmaceutical composition as tablets, pills, dragees, capsules, gel capsules, liquids, gels, syrups, slurries, suspensions, and the like, for oral ingestion by a subject.

In embodiments, suitable pharmaceutically acceptable carriers may be solid or liquid carriers, or liquid excipients. Such pharmaceutically acceptable liquid carriers can be aqueous or non-aqueous solutions, suspensions, and emulsions. Liquid carriers suitable for use in accordance with the present disclosure can be used in preparing solutions, suspensions, emulsions, syrups, elixirs, and pressurized compounds. The active ingredient (e.g., one or more compounds of the present disclosure) can be dissolved or suspended in a pharmaceutically acceptable liquid carrier such as water, an organic solvent, a mixture of both or pharmaceutically acceptable oils or fats. In some embodiments, the liquid carrier further comprises other suitable pharmaceutical additives such as solubilizers, emulsifiers, buffers, preservatives, sweeteners, flavoring agents, suspending agents, thickening agents, colors, viscosity regulators, stabilizers, or osmo-regulators.

In embodiments, the pharmaceutical composition comprises an aqueous carrier. Aqueous carriers suitable for use in accordance with the present disclosure include, but are not limited to, water, ethanol, alcoholic/aqueous solutions, glycerol, emulsions, or suspensions, including saline and buffered media.

Examples of non-aqueous solvents suitable for use in accordance with the present disclosure include, but are not limited to, propylene glycol, polyethylene glycol, vegetable oils such as olive oil, and injectable organic esters such as ethyl oleate.

Liquid pharmaceutical compositions may be prepared using compounds of the present disclosure, and any other solid excipients where the components are dissolved or suspended in a liquid carrier such as water, vegetable oil, alcohol, polyethylene glycol, propylene glycol, or glycerin.

Liquid pharmaceutical compositions may contain emulsifying agents to disperse uniformly throughout the composition and/or combination an active ingredient or other excipient that is not soluble in the liquid carrier. Emulsifying agents that may be useful in liquid compositions and/or combinations of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

A liquid composition can also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

In embodiments, the pharmaceutical composition comprises a solid carrier. Solid carriers suitable for use in accordance with the present disclosure include, but are not limited to, inert substances such as lactose, starch, glucose, methyl-cellulose, magnesium stearate, dicalcium phosphate, mannitol, and the like.

In embodiments, the solid compositions further comprise one or more substances acting as flavoring agents, lubricants, solubilizers, suspending agents, fillers, glidants, compression aids, binders, or tablet-disintegrating agents. In powders, the carrier can be a finely divided solid which is in admixture with the finely divided active ingredient. In tablets, the active ingredient is mixed with a carrier having suitable compression properties in suitable proportions and compacted in the shape and size desired. The powders and tablets may contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

In embodiments, a tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, disintegrant, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In embodiments, the pharmaceutical composition comprises one or more diluents. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

In embodiments, the pharmaceutical composition comprises one or more disintegrants. The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

In embodiments, the pharmaceutical composition comprises one or more glidants. Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

In embodiments, the pharmaceutical composition comprises one or more flavoring agents and/or flavor enhancers. Flavoring agents and flavor enhancers make the dosage form more palatable to the patient. Common flavoring agents and flavor enhancers for pharmaceutical products that may be included in the pharmaceutical composition include maltol, vanillin, ethyl vanillin, menthol, citric acid, fumaric acid, ethyl maltol, and tartaric acid.

In embodiments, the pharmaceutical composition comprises one or more sweetening agents. Sweetening agents such as aspartame, lactose, sorbitol, saccharin, sodium saccharin, sucrose, aspartame, fructose, mannitol, and invert sugar may be added to improve the taste.

In embodiments, the pharmaceutical composition comprises one or more of a preservative and/or chelating agents. Preservatives and chelating agents such as alcohol, sodium benzoate, butylated hydroxyl toluene, butylated hydroxyanisole, and ethylenediamine tetraacetic acid may be added at levels safe for ingestion to improve storage stability.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

When a dosage form such as a tablet is made by the compaction of a powdered composition, the composition is subjected to pressure from a punch and dye. Some excipients and active ingredients tend to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Solid and liquid compositions may also be dyed using any pharmaceutically acceptable colorant to improve their appearance and/or facilitate patient identification of the product and unit dosage level.

In certain embodiments, a pharmaceutical composition of the present disclosure is prepared using known techniques, including, but not limited to mixing, dissolving, granulating, dragee-making, levigating, emulsifying, encapsulating, entrapping, or tableting processes.

### Liquid Dosage Forms

In embodiments, the amount of CBD in the pharmaceutical composition may range from about 1 mg/mL to about 100 mg/mL, for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein. In some embodiment, the pharmaceutical composition comprises about 20 mg/mL to about 30 mg/mL of CBD (e.g., about 20 mg/mL, about 20.5 mg/mL, about 21 mg/mL, about 21.5 mg/mL, about 22 mg/mL, about 22.5 mg/mL, about 23 mg/mL, about 23.5 mg/mL, about 24 mg/mL, about 24.5 mg/mL, about 25 mg/mL, about 25.5 mg/mL, about 26 mg/mL, about 26.5 mg/mL, about 27 mg/mL, about 27.5 mg/mL, about 28 mg/mL, about 28.5 mg/mL, about 29 mg/mL, about 29.5 mg/mL, about 30 mg/mL, including all values and ranges therein). In embodiments, the pharmaceutical composition comprises 22.5 mg/mL to about 27.5 mg/mL of CBD. In embodiments, the pharmaceutical composition comprises 23.7 mg/mL to about 26.5 mg/mL of CBD.

In embodiments, the amount of THC in the pharmaceutical composition can range from about 1 mg/mL to about 100 mg/mL, for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein. In embodiments, the pharmaceutical composition comprises about 20 mg/mL to about 30 mg/mL of THC (e.g., about 20 mg/mL, about 20.5 mg/mL, about 21 mg/mL, about 21.5 mg/mL, about 22 mg/mL, about 22.5 mg/mL, about 23 mg/mL, about 23.5 mg/mL, about 24 mg/mL, about 24.5 mg/mL, about 25 mg/mL, about 25.5 mg/mL, about 26 mg/mL, about 26.5 mg/mL, about 27 mg/mL, about 27.5 mg/mL, about 28 mg/mL, about 28.5 mg/mL, about 29 mg/mL, about 29.5 mg/mL, about 30 mg/mL, including all values and ranges therein). In embodiments, the pharmaceutical composition comprises about 22.5 mg/mL to about 27.5 mg/mL of THC. In embodiments, the pharmaceutical composition comprises about 24.3 mg/ml to about 28.4 mg/mL of THC.

In embodiments, the pharmaceutical composition comprises about 1 mg/mL to about 100 mg/mL of THC (for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein), and about 1 mg/mL to about 100 mg/mL of CBD, for example, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, including all values and ranges therein.

In embodiments, the pharmaceutical composition comprises about 20-30 mg/mL THC and about 20-30 mg/mL CBD. In embodiments, the pharmaceutical composition comprises about 22.5 mg/mL to about 27.5 mg/mL of THC (for example, 22.5 mg/mL, 22.6 mg/mL, 22.7 mg/mL, 22.8 mg/mL, 22.9 mg/mL, 23.0 mg/mL, 23.1 mg/mL, 23.2 mg/mL, 23.3 mg/mL, 23.4 mg/mL, 23.5 mg/mL, 23.6 mg/mL, 23.7 mg/mL, 23.8 mg/mL, 23.9 mg/mL, 24.0 mg/mL, 24.1 mg/mL, 24.2 mg/mL, 24.3 mg/mL, 24.4 mg/mL, 24.5 mg/mL, 24.6 mg/mL, 24.7 mg/mL, 24.8 mg/mL, 24.9 mg/mL, 25.0 mg/mL, 25.1 mg/mL, 25.2 mg/mL, 25.3 mg/mL, 25.4 mg/mL, 25.5 mg/mL, 25.6 mg/mL, 25.7 mg/mL, 25.8 mg/mL, 25.9 mg/mL, 26.0 mg/mL, 26.1 mg/mL, 26.2 mg/mL, 26.3 mg/mL, 26.4 mg/mL, 26.5 mg/mL, 26.6 mg/mL, 26.7 mg/mL, 26.8 mg/mL, 26.9 mg/mL, 27.0 mg/mL, 27.1 mg/mL, 27.2 mg/mL, 27.3 mg/mL, 27.4 mg/mL, 27.5 mg/mL, including all values and ranges therein), and about 22.5 mg/mL to about 27.5 mg/mL of CBD (for example, 22.5 mg/mL, 22.6 mg/mL, 22.7 mg/mL, 22.8 mg/mL, 22.9 mg/mL, 23.0 mg/mL, 23.1 mg/mL, 23.2 mg/mL, 23.3 mg/mL, 23.4 mg/mL, 23.5 mg/mL, 23.6 mg/mL, 23.7 mg/mL, 23.8 mg/mL, 23.9 mg/mL, 24.0 mg/mL, 24.1 mg/mL, 24.2 mg/mL, 24.3 mg/mL, 24.4 mg/mL, 24.5 mg/mL, 24.6 mg/mL, 24.7 mg/mL, 24.8 mg/mL, 24.9 mg/mL, 25.0 mg/mL, 25.1 mg/mL, 25.2 mg/mL, 25.3 mg/mL, 25.4 mg/mL, 25.5 mg/mL, 25.6 mg/mL, 25.7 mg/mL, 25.8 mg/mL, 25.9 mg/mL, 26.0 mg/mL, 26.1 mg/mL, 26.2 mg/mL, 26.3 mg/mL, 26.4 mg/mL, 26.5 mg/mL, 26.6 mg/mL, 26.7 mg/mL, 26.8 mg/mL, 26.9 mg/mL, 27.0 mg/mL, 27.1 mg/mL, 27.2 mg/mL, 27.3 mg/mL, 27.4 mg/mL, 27.5 mg/mL, including all values and ranges therein). In embodiments, the pharmaceutical composition comprises about 22.5 mg/mL to about 27.5 mg/mL of THC, and about 22.5 mg/mL to about 27.5 mg/mL of CBD. In embodiments, the pharmaceutical composition comprises about 24.3 mg/mL to about 28.4 mg/mL of THC, and about 23.7 mg/mL to about 26.5 mg/mL of CBD. In embodiments, the pharmaceutical composition comprises about 27 mg/mL of THC and about 25 mg/mL of CBD.

In embodiments, the THC and CBD are present in a ratio by weight of from 0.5:1.5 to 1.5:0.5. In embodiments, the THC and CBD are present in a ratio by weight of from 0.7:1.3 to 1.3:0.7. In embodiments, the THC and CBD are present in a ratio by weight of from 0.9:1.1 to 1.1:0.9. In embodiments, the THC and CBD are present in a ratio by weight of 1.1:0.9.

As discussed herein, in some embodiments, the pharmaceutical composition further comprises (in addition to CBD and THC, and optionally one or more additional cannabinoids described herein) one or more terpenes. In embodiments, the total terpene content in the pharmaceutical composition ranges from 1.1 mg/mL to 8.3 mg/mL (e.g., 1.1 mg/mL mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.3 mg/mL, including all values therein). In embodiments, the pharmaceutical composition further comprises 0.05-1.6 mg/mL Myrcene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, including all values therein), 0.1-5.0 mg/mL Trans-Caryophyllene (for example, 0.1 mg/mL, 0.2 mg/mL, 0.3 mg/mL, 0.4 mg/mL, 0.5 mg/mL, 1 mg/mL, 1.5 mg/mL, 2 mg/mL, 2.5 mg/mL, 3 mg/mL, 3.1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mL, 4.0 mg/mL, 4.1 mg/mL, 4.2 mg/mL, 4.3 mg/mL, 4.4 mg/mL, 4.5 mg/mL, 4.6 mg/mL, 4.7 mg/mL, 4.8 mg/mL, 4.9 mg/mL, 5.0 mg/mL, including all values therein), 0.05-1.0 mg/mL Alpha-Bergamotene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-1.0 mg/mL Beta-Farnesene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-2.0 mg/mL Alpha-Caryophyllene (for example, 0.05 mg/mL, 0.1 mg/mL, 0.2 mg/mL, 0.3 mg/mL, 0.4 mg/mL, 0.5 mg/mL, 1.0 mg/mL, 1.5 mg/mL, 2.0 mg/mL, including all values therein), 0.05-1.0 mg/mL Guaia-3,9-diene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-1.0 mg/mL Selina-3,7(11)-diene (for example, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.1-1.0 mg/mL Trans-Nerolidol (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, including all values therein), 0.05-0.5 mg/mL Alpha-Bisabolol (for example, 0.05 mg/mL, 0.1 mg/mL, 0.2 mg/mL, 0.3 mg/mL, 0.4 mg/mL, 0.5 mg/mL, including all values therein), 0.1-1.5 mg/mL Trans-Phytol (for example, 0.1 mg/mL, 0.2 mg/mL, 0.3 mg/mL, 0.4 mg/mL, 0.5 mg/mL, 0.6 mg/mL, 0.7 mg/mL, including all values therein). In embodiments, the pharmaceutical composition further comprises no more than 0.8 mg/mL Myrcene, 0.1-2.8 mg/mL Trans-Caryophyllene, not more than 0.3 mg/mL Alpha-Bergamotene, not more than 0.5 mg/mL Beta-Farnesene, 0.1-1.1 mg/mL Alpha-Caryophyllene, not more than 0.5 mg/mL Guaia-3,9-diene, nor more than 0.6 mg/mL Selina-3,7(11)-diene, 0.1-0.4 mg/mL Trans-Nerolidol, not more than 0.3 mg/mL Alpha-Bisabolol, 0.1-0.7 mg/mL Trans-Phytol.

In embodiments, the pharmaceutical composition comprises components listed in Table 3.

TABLE 3
Example composition

| Com- | | | | | |
|---|---|---|---|---|---|
| ponent | Concentration | | Component | Concentration | |
| THC | 24.3-28.4 | mg/mL | Myrcene | NMT 0.8 | mg/mL |
| CBD | 23.7-26.5 | mg/mL | Trans- | 0.1-2.8 | mg/mL |
| | | | Caryophyllene | | |
| OH- | 0.1-0.4 | mg/mL | Alpha- | NMT 0.3 | mg/mL |
| CBD | | | Bergamotene | | |
| CBDV | 0.1-0.3 | mg/mL | Beta-Farnesene | NMT 0.5 | mg/mL |
| THCV | 0.1-0.4 | mg/mL | Alpha- | 0.1-1.1 | mg/mL |
| | | | Caryophyllene | | |
| CBG | 0.2-1.6 | mg/mL | Guaia- | NMT 0.5 | mg/mL |
| | | | 3,9-diene | | |
| CBN | 0.1-0.9 | mg/mL | Selina-3,7 | NMT 0.6 | mg/mL |
| | | | (11)-diene | | |
| DHC | NMT 0.3 | mg/mL | Trans- | 0.1-0.4 | mg/mL |
| | | | Nerolidol | | |
| CBC | 1.0-2.8 | mg/mL | Alpha- | NMT 0.3 | mg/mL |
| | | | Bisabolol | | |
| CBG | NMT 0.4 | mg/mL | Trans-Phytol | 0.1-0.7 | mg/mL |
| MME | | | | | |

NMT: Not More Than

In embodiments, the pharmaceutical composition is an oromucosal spray. In embodiments, the oromucosal spray releases or delivers about 50 μL to about 200 μL (e.g., about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, or about 200 μL, including all values and ranges therein) per spray. In embodiments, the oromucosal spray releases or delivers about 100 μL per spray. In embodiments, the oromucosal spray releases or delivers about 2 to about 3 mg of THC per spray (for example, 2.00 mg, 2.01 mg, 2.02 mg, 2.03 mg, 2.04 mg, 2.05 mg, 2.06 mg, 2.07 mg, 2.08 mg, 2.09 mg, 2.10 mg, 2.11 mg, 2.12 mg, 2.13 mg, 2.14 mg, 2.15 mg, 2.16 mg, 2.17 mg, 2.18 mg, 2.19 mg, 2.20 mg, 2.21 mg, 2.22 mg, 2.23 mg, 2.24 mg, 2.25 mg, 2.26 mg, 2.27 mg, 2.28 mg, 2.29 mg, 2.30 mg, 2.31 mg, 2.32 mg, 2.33 mg, 2.34 mg, 2.35 mg, 2.36 mg, 2.37 mg, 2.38 mg, 2.39 mg, 2.40 mg, 2.41 mg, 2.42 mg, 2.43 mg, 2.44 mg, 2.45 mg, 2.46 mg, 2.47 mg, 2.48 mg, 2.49 mg, 2.50 mg, 2.51 mg, 2.52 mg, 2.53 mg, 2.54 mg, 2.55 mg, 2.56 mg, 2.57 mg, 2.58 mg, 2.59 mg, 2.60 mg, 2.61 mg, 2.62 mg, 2.63 mg, 2.64 mg, 2.65 mg, 2.66 mg, 2.67 mg, 2.68 mg, 2.69 mg, 2.70 mg, 2.71 mg, 2.72 mg, 2.73 mg, 2.74 mg, 2.75 mg, 2.76 mg, 2.77 mg, 2.78 mg, 2.79 mg, 2.80 mg, 2.81 mg, 2.82 mg, 2.83 mg, 2.84 mg, 2.85 mg, 2.86 mg, 2.87 mg, 2.88 mg, 2.89 mg, 2.90 mg, 2.91 mg, 2.92 mg, 2.93 mg, 2.94 mg, 2.95 mg, 2.96 mg, 2.97 mg, 2.98 mg, 2.99 mg, 3.00 mg, including all values and ranges therein), and about 2 to about 3 mg of CBD per spray (for example, 2.25 mg, 2.26 mg, 2.27 mg, 2.28 mg, 2.29 mg, 2.30 mg, 2.31 mg, 2.32 mg, 2.33 mg, 2.34 mg, 2.35 mg, 2.36 mg, 2.37 mg, 2.38 mg, 2.39 mg, 2.40 mg, 2.41 mg, 2.42 mg, 2.43 mg, 2.44 mg, 2.45 mg, 2.46 mg, 2.47 mg, 2.48 mg, 2.49 mg, 2.50 mg, 2.51 mg, 2.52 mg, 2.53 mg, 2.54 mg, 2.55 mg, 2.56 mg, 2.57 mg, 2.58 mg, 2.59 mg, 2.60 mg, 2.61 mg, 2.62 mg, 2.63 mg, 2.64 mg, 2.65 mg, 2.66 mg, 2.67 mg, 2.68 mg, 2.69 mg, 2.70 mg, 2.71 mg, 2.72 mg, 2.73 mg, 2.74 mg, 2.75 mg, 2.76 mg, 2.77 mg, 2.78 mg, 2.79 mg, 2.80 mg, 2.81 mg, 2.82 mg, 2.83 mg, 2.84 mg, 2.85 mg, 2.86 mg, 2.87 mg, 2.88 mg, 2.89 mg, 2.90 mg, 2.91 mg, 2.92 mg, 2.93 mg, 2.94 mg, 2.95 mg, 2.96 mg, 2.97 mg, 2.98 mg, 2.99 mg, 3.00 mg, including all values and ranges therein). In embodiments, the oromucosal spray releases or delivers about 2.25 mg to about 2.75 mg of THC (e.g., about 2.25 mg, about 2.3 mg, about 2.35 mg, about 2.4 mg, about 2.45 mg, about 2.5 mg, about 2.55 mg, about 2.65 mg, about 2.7 mg, or about 2.75 mg) and about 2.25 to about 2.75 mg of CBD (e.g., about 2.25 mg, about 2.3 mg, about 2.35 mg, about 2.4 mg, about 2.45 mg, about 2.5 mg, about 2.55 mg, about 2.65 mg, about 2.7 mg, or about 2.75 mg). In embodiments, the oromucosal spray releases or delivers about 2.7 mg of THC and about 2.5 mg of CBD.

The oromucosal spray described herein may contain any suitable pharmaceutically acceptable excipient. In embodiments, the oromucosal spray comprises a pharmaceutically acceptable solvent suitable to dissolve cannabinoids (e.g., THC and CBD). In embodiments, the solvent comprises a C1-C4 alcohol. In embodiments, the solvent comprises ethanol. The solvent and co-solvent may be present in an appropriate amount to solubilize the cannabinoids and allow delivery of the cannabinoids to the oral cavity via a spray. In embodiments, the solvent is present in an amount ranging from about 50%-98% w/w based on the total weight of the oromucosal spray (e.g., about 50%, about 55%, about 60%, about 65%, about 70%, about 75%, about 80%, about 85%, about 90%, about 91%, about 92%, about 93%, about 94%, about 95%, about 96%, about 97%, or about 98% v/v, based on the total weight of the oromucosal spray). In embodiments, the solvent is present in amount of at least about 65% w/w. In embodiments, the solvent is present in amount of at least about 70% w/w. In embodiments, the solvent is present in amount of at least about 75% w/w. In embodiments, the solvent is present in amount of at least about 80% w/w. In embodiments, the solvent is present in amount of at least about 85% w/w. In embodiments, the solvent is present in amount ranging from 80% w/w to about 98% w/w of the formulation.

In embodiments, the oromucosal spray further a pharmaceutically acceptable co-solvent. In embodiments, co-solvent is a glycol, sugar alcohol, carbonate ester or chlorinated hydrocarbons. In embodiments, co-solvent is a glycol. In embodiments, the glycerol is propylene glycol or glycerol. In embodiments, the glycerol is propylene glycol. In embodiments, the co-solvent is a carbonate ester. In embodiments, the carbonate ester is propylene carbonate. In embodiments, the co-solvent is propylene glycol. In embodiments, the solvent is ethanol, and the co-solvent is propylene glycol.

In embodiments, the solvent and co-solvent are present in a weight ratio ranging from 60/40 to 40/60 (e.g., 60/40, 59/41, 58/42, 57/43, 56/44, 55/45, 54/46, 53/47, 52/48, 51/49, 50/50, 49/51, 48/52, 47/53, 46/54, 45/55, 44/56, 43/57, 42/58, 41/59, 40/60, including all subranges therein). In embodiments, the solvent and co-solvent are present in a weight ratio in the range 55/45 to 45/55. In embodiments, the solvent and co-solvent are present in a weight ratio of about 50/50.

In embodiments, the solvent is ethanol and the co-solvent is propylene glycol. In embodiments, ethanol/propylene glycol are present in weight ratio ranging from 55/45 to 45/55. In embodiments, ethanol/propylene glycol are present in a weight ratio of about 50/50.

The solvent and co-solvent may be present in an appropriate amount to solubilize the cannabinoids and allow delivery of the cannabinoids to the oral cavity via a spray. In embodiments, the solvent and co-solvent are present in an amount ranging from about 50%-98% w/w based on the total weight of the oromucosal spray (e.g., about 50%, about 55%, about 60%, about 65%, about 70%, about 75%, about 80%, about 85%, about 90%, about 91%, about 92%, about 93%, about 94%, about 95%, about 96%, about 97%, or about 98% v/v, based on the total weight of the oromucosal spray). In embodiments, the solvent and co-solvent are present in amount of at least about 65% w/w. In embodiments, the solvent and co-solvent are present in amount of at least about 70% w/w. In embodiments, the solvent and co-solvent are present in amount of at least about 75% w/w. In embodiments, the solvent and co-solvent are present in amount of at least about 80% w/w. In embodiments, the solvent and co-solvent are present in amount of at least about 85% w/w. In embodiments, the solvent and co-solvent are present in amount ranging from 80% w/w to about 98% w/w of the formulation.

In embodiments, the oromucosal spray comprises flavoring. In embodiments, the flavoring is strawberry, cherry, peppermint, orange, grape, or other flavors. In embodiments, the flavoring is peppermint oil. In embodiments, the flavoring is present in a suitable amount such that the formulation is palatable. In embodiments, the flavoring is present in a suitable amount to mask the taste of the cannabinoids. In embodiments, the flavoring is present in an amount up to 0.1% v/v based on the total volume of the oromucosal spray, e.g., 0.01, 0.02, 0.03, 0.04, 0.05, 0.06, 0.07, 0.08, 0.09, or 0.1% v/v, including all values and ranges therein.

Non-limiting examples of an oromucosal spray comprising CBD and THC of the present disclosure are described in U.S. Pat. No. 8,603,515 (Published May 2, 2013), which is incorporated by reference its entirety herein.

In embodiments, the oromucosal spray comprises THC and CBD (e.g., in the amounts described herein), and one or more of OH-CBD, CBDV, THCV, CBG, CBN, DHC, CBC, mono-methylated CBG (CBG MME), or any combination thereof. In embodiments, the oromucosal spray comprises about 20-30 mg/mL THC (for example, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, including all values therein), 20-30 mg/mL CBD (for example, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, including all values therein), 0.1-0.8 mg/mL OH-CBD (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, including all values therein), 0.1-0.6 mg/mL CBDV (for example, 0.1 mg/mL, 0.2 mg/mL, 0.3 mg/mL, 0.4 mg/mL, 0.5 mg/mL, 0.6 mg/mL, including all values therein), 0.1-0.8 mg/mL THCV (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, including all values therein), 0.2-3.2 mg/mL CBG (for example, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3.0 mg/mL, 3.1 mg/mL, 3.2 mg/mL, including all values therein), 0.1-1.8 mg/mL CBN (for example, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 0.1 mg/mL, 1.2 mg/mL, 1.3 mg/mL, 1.4 mg/mL, 1.5 mg/mL, 1.6 mg/mL, 1.7 mg/mL, 1.8 mg/mL, including all values therein), 0.05-0.3 mg/mL DHC (for example, 0.05 mg/mL, 0.1 mg/mL, 0.2 mg/mL, 0.3 mg/mL, including all values therein), 1.0-5.0 mg/mL CBC (for example, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3.0 mg/mL, 3.1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mL, 4.0 mg/mL, 4.1 mg/mL, 4.2 mg/mL, 4.3 mg/mL, 4.4 mg/mL, 4.5 mg/mL, 4.6 mg/mL, 4.7 mg/mL, 4.8 mg/mL, 4.9 mg/mL, 5.0 mg/mL, including all values therein), and 0.05-0.4 mg/mL CBG MME (for example, 0.05 mg/mL, 0.1 mg/mL, 0.2 mg/mL, 0.3 mg/mL, 0.4 mg/mL, including all values therein). In embodiments, the oromucosal spray comprises 24.3-28.4 mg/mL THC, 23.7-26.5 mg/mL CBD, 0.1-0.4 mg/mL OH-CBD, 0.1-0.3 mg/mL CBDV, 0.1-0.4 mg/mL THCV, 0.2-1.6 mg/mL CBG, 0.1-0.9 mg/mL CBN, 0.05-0.3 mg/mL DHC, 1.0-2.8 mg/mL CBC, and 0.05-0.4 mg/mL CBG MME.

### Methods of Treatment

In embodiments, the drug substance (e.g., botanical drug substance comprising THC and CBD) is administered at a dose of from about 0.001 mg/kg to about 100 mg/kg, for example, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg, including all values and ranges therein.

In embodiments, the pharmaceutical composition is administered such that THC is administered at a dose of from about 0.001 mg/kg to about 100 mg/kg, for example, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg, including all values and ranges therein. In embodiments, THC is administered at a dose of from about 0.05 mg/kg to about 25 mg/kg. In embodiments, THC is administered at a dose of from about 0.1 mg/kg to about 12.5 mg/kg. In embodiments, THC is administered at a dose of from about 0.2 mg/kg to about 7.5 mg/kg. In embodiments, THC is administered at a dose of from about 2.7 mg/kg. In embodiments, THC is administered at a dose of from about 5.4 mg/kg. In embodiments, THC is administered at a dose of from about 10.8 mg/kg.

In embodiments, the pharmaceutical composition is administered such that CBD is administered at a dose of from about 0.001 mg/kg to about 100 mg/kg, for example, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg, including all values and ranges therein. In embodiments, CBD is administered at a dose of from about 0.05 mg/kg to about 25 mg/kg. In embodiments, CBD is administered at a dose of from about 0.1 mg/kg to about 12.5 mg/kg. In embodiments, CBD is administered at a dose of from about 0.2 mg/kg to about 7.5 mg/kg. In embodiments, THC is administered at a dose of from about 2.5 mg/kg. In embodiments, THC is administered at a dose of from about 5 mg/kg. In embodiments, THC is administered at a dose of from about 10 mg/kg.

In embodiments, the pharmaceutical composition is administered such that THC is administered at a dose of from about 0.001 mg/kg to about 100 mg/kg (for example, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg, including all values and ranges therein) and CBD is administered at a dose of from about 0.001 mg/kg to about 100 mg/kg (for example, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg, including all values and ranges therein). In embodiments, THC is administered at a dose of from about 0.05 mg/kg to about 25 mg/kg and CBD is administered at a dose of from about 0.05 mg/kg to about 25 mg/kg. In embodiments, THC is administered at a dose of from about 0.1 mg/kg to about 12.5 mg/kg and CBD is administered at a dose of from about 0.1 mg/kg to about 12.5 mg/kg. In embodiments, THC is administered at a dose of from about 0.2 mg/kg to about 7.5 mg/kg and CBD is administered at a dose of from about 0.2 mg/kg to about 7.5 mg/kg.

In embodiments, THC and CBD are administered at a total dose of from about 0.001 mg/kg to about 100 mg/kg, for example, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg, including all values and ranges therein. In embodiments, THC and CBD are administered at a total dose of from about 0.05 mg/kg to about 25 mg/kg. In embodiments, the pharmaceutical composition is administered such that THC and CBD are administered at a total dose of from about 0.1 mg/kg to about 12.5 mg/kg. In embodiments, THC and CBD are administered at a total dose of from about 0.2 mg/kg to about 7.5 mg/kg. In embodiments, THC and CBD are administered at a total dose of about 5.2 mg/kg. In embodiments, THC and CBD are administered at a total dose of about 10.4 mg/kg. In embodiments, THC and CBD are administered at a total dose of about 20.8 mg/kg.

In embodiments, the pharmaceutical composition is administered in combination (e.g., as an adjunctive treatment) with an additional form of treatment. Non-limiting examples of the additional form of treatment include alcohols, antibiotics, anticholinergics, anticonvulsants, antidepressants, antihypertensives, antiparkinsonian agents, antipsychotics, octanoic acid, benzodiazepines, beta blockers, botulinum toxin, bronchodilators, diuretics, CBD1/CB2 agonists and antagonists, lifestyle changes, selective GABAB receptor agonists, surgery, therapy, transcranial magnetic stimulation, ultrasound, and combinations thereof. Non-limiting examples of alcohols include ethanol and octanol (e.g., 1-octanol). Non-limiting examples of antibiotics include isoniazid. Non-limiting examples of anticonvulsants include carisbamate, pregabalin (Lyrica), topiramate, gabapentin, pregabalin, levetiracetam, and phenobarbital. Non-limiting examples of antidepressants include mirtazapine. Non-limiting examples of antihypertensives include nimodipine. Non-limiting examples of antiparkinsonian agents include levodopa, zonisamide, and carbidopa. Non-limiting examples of antipsychotics includes clozapine, olanzapine. Non-limiting examples of benzodiazepines include clonazepam and alprazolam. Non-limiting examples of beta blockers include propranolol, atenolol, nadolol, metoprolol, timolol, sotalol, and primidone. Non-limiting examples of bronchodilators include theophylline. Non-limiting examples of CBD1/CB2 agonists and antagonists include WIN55,212-2 and AM251. Non-limiting examples diuretics include acetazolamide. Non-limiting examples of lifestyle changes include elimination or reduction of tobacco use. Non-limiting examples of selective GABAB receptor agonists include R-baclofen. Non-limiting examples of surgery include thalamotomy and pallidotomy. Non-limiting examples of therapy include physical therapy, speech-language therapy, occupational therapy, and deep brain stimulation therapy. In embodiments, the pharmaceutical composition may be administered sequentially, concurrently, or simultaneously with the additional form of treatment.

In embodiments, the pharmaceutical composition is administered as an oromucosal spray described herein. In embodiments, from about 1 to about 20 sprays per day of the oromucosal spray are administered. In embodiments, about 1 spray per day of the oromucosal spray is administered. In embodiments, about 2 sprays per day of the oromucosal spray are administered. In embodiments, about 3 sprays per day of the oromucosal spray are administered. In embodiments, about 4 sprays per day of the oromucosal spray are administered. In embodiments, about 5 sprays per day of the oromucosal spray are administered. In embodiments, about 6 sprays per day of the oromucosal spray are administered. In embodiments, about 7 sprays per day of the oromucosal spray are administered. In embodiments, about 8 sprays per day of the oromucosal spray are administered. In embodiments, about 9 sprays per day of the oromucosal spray are administered. In embodiments, about 10 sprays per day of the oromucosal spray are administered. In embodiments, about 11 sprays per day of the oromucosal spray are administered. In embodiments, about 12 sprays per day of the oromucosal spray are administered. In embodiments, about 13 sprays per day of the oromucosal spray are administered. In embodiments, about 14 sprays per day of the oromucosal spray are administered. In embodiments, about 15 sprays per day of the oromucosal spray are administered. In embodiments, about 16 sprays per day of the oromucosal spray are administered. In embodiments, about 17 sprays per day of the oromucosal spray are administered. In embodiments, about 18 sprays per day of the oromucosal spray are administered. In embodiments, about 19 sprays per day of the oromucosal spray are administered. In embodiments, about 20 sprays per day of the oromucosal spray are administered.

In embodiments, the methods disclosed herein comprise a time interval or “gap” between administering each spray. In embodiments, the gap between sprays is at least 15 minutes. In embodiments, the gap is between sprays is 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 90 minutes, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, 24 hours, or any time intervals or ranges in between. In embodiments, the initiation spray dosing is evenly spread out throughout the day.

In embodiments, the pharmaceutical composition is administered for a defined length of time, including the titration period. In embodiments, the pharmaceutical composition is administered for at least about 14 days e.g., 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 24 weeks, or 25 weeks, or 26 weeks, or 27 weeks, or 28 weeks, or 29 weeks, or 30 weeks, or 31 weeks, or 32 weeks, or 33 weeks, or 34 weeks, or 35 weeks, or 36 weeks, or 37 weeks, or 38 weeks, or 39 weeks, or 40 weeks, or 41 weeks, or 42 weeks, or 43 weeks, or 44 weeks, or 45 weeks, or 46 weeks, or 47 weeks, or 48 weeks, or 49 weeks, or 50 weeks, or 51 weeks, or 52 weeks, or 53 weeks, or 54 weeks, or 55 weeks, or 56 weeks, or 57 weeks, or 58 weeks, or 59 weeks, or 60 weeks, or 61 weeks, or 62 weeks, or 63 weeks, or 64 weeks, or 65 weeks, or 66 weeks, or 67 weeks, or 68 weeks, or 69 weeks, or 70 weeks, or 71 weeks, or 72 weeks, or 73 weeks, or 74 weeks, or 75 weeks, or 76 weeks, or 77 weeks, or 78 weeks, or 79 weeks, or 80 weeks, or 81 weeks, or 82 weeks, or 83 weeks, or 84 weeks, or 85 weeks, or 86 weeks, or 87 weeks, or 88 weeks, or 89 weeks, or 90 weeks, or 91 weeks, or 92 weeks, or 93 weeks, or 94 weeks, or 95 weeks, or 96 weeks, or 97 weeks, or 98 weeks, or 99 weeks, or 100 weeks, or 101 weeks, or 102 weeks, or 103 weeks, or 104 weeks, 3 years, 4 years, 5 years, 6 years, 7 years, 8 years, 9 years, 10 years, 11 years, 12, years, 13 years, 14 years, 15 years, 16 years, 17 years, 18 years, 19 years, 20 years, or more.

### Clinical Outcomes

The methods of this disclosure are used to treat patients with tremor. In embodiments, the methods comprise treating ET. In embodiments, the treating comprises reducing tremor intensity.

In embodiments, efficacy is determined by measuring tremor frequency or average tremor frequency after treatment. In embodiments, the tremor after treatment has a lower frequency or average frequency than before treatment. In embodiments, the tremor after treatment has a frequency or average frequency of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25 or more Hz, one or more of any value therein or range therebetween, or combinations thereof, as measured during one or more tremor events. In embodiments, the tremor after treatment has a frequency or average frequency of about 4 to 12 Hz, as measured during one or more tremor events. In embodiments, the tremor after treatment has a frequency or average frequency that is reduced compared to a control by about 10% to about 100%, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or any value or range therein. In embodiments, the tremor after treatment has a frequency or average frequency that is reduced by about 1, 2, 3, 4, 5, 6, 7, 8, 9, 10, 11, 12, 13, 14, 15, 16, 17, 18, 19, 20, 21, 22, 23, 24, or 25 Hz, or any value or range therein.

In embodiments, efficacy is determined by measuring tremor amplitude or average tremor amplitude after treatment. In embodiments, the tremor after treatment has an amplitude or average amplitude of less than or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 dBV, one or more of any value or range therein, or combinations thereof, as measured during one or more tremor events. In embodiments, treatment reduces tremor amplitude or average tremor amplitude compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or more dBV, one or more of any value therein or range therebetween, or combinations thereof, as measured during one or more tremor events.

In embodiments, the tremor after treatment has an amplitude or average amplitude of about 1 or less, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, less than 300 mm, one or more of any value therein or range therebetween, or combinations thereof, as measured during one or more tremor events. In embodiments, treatment reduces tremor amplitude or average tremor amplitude compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or more mm, one or more of any value therein or range therebetween, or combinations thereof, as measured during one or more tremor events.

In embodiments, treatment reduces tremor amplitude or average tremor amplitude compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, one or more of any value therein or range therebetween, or combinations thereof, as measured during one or more tremor events.

In embodiments, efficacy is determined by scoring on the observational scoring scale described herein (and summarized in TABLE A below) after treatment. In embodiments, the tremor after treatment has an observational score (“OS”) of 0, 1, 2, or 3. In embodiments, treatment reduces OS by 1, 2, 3, or 4 points compared to a control.

In embodiments, efficacy is determined by measuring Tremor Index (“TI”) or average TI after treatment. In embodiments, the tremor after treatment has a TI or average TI of about −100, −99, −98, −97, −96, −95, −94, −93, −92, −91, −90, −89, −88, −87, −86, −85, −84, −83, −82, −81, −80, −79, −78, −77, −76, −75, −74, −73, −72, −71, −70, −69, −68, −67, −66, −65, −64, −63, −62, −61, −60, −59, −58, −57, −56, −55, −54, −53, −52, −51, −50, −49, −48, −47, −46, −45, −44, −43, −42, −41, −40, −39, −38, −37, −36, −35, −34, −33, −32, −31, −30, −29, −28, −27, −26, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, less than 100 dBV, one or more of any value therein or range therebetween, or combinations thereof, as measured during one or more tremor events. In embodiments, treatment reduces TI or average TI compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 dBV, one or more of any value therein or range therebetween, or combinations thereof, as measured during one or more tremor events. In embodiments, treatment reduces TI or average TI compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200%, one or more of any value therein or range therebetween, or combinations thereof, as measured during one or more tremor events.

In embodiments, efficacy is determined by measuring Motion Power Percentage (“MPP”) or average MPP after treatment. In embodiments, the tremor after treatment has an MPP or average MPP of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, less than 100%, one or more of any value therein or range therebetween, or combinations thereof, as measured during one or more tremor events. In embodiments, the tremor after treatment has an MPP or average MPP of less than about 40 to 80%, one or more of any value or range therein, or combinations thereof, as measured during one or more tremor events. In embodiments, treatment reduces MPP or average MPP compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, one or more of any value therein or range therebetween, or combinations thereof, as measured during one or more tremor events.

In embodiments, efficacy is determined by measuring the number of tremor events during a 5 minute period after treatment. In embodiments, the tremor after treatment results in 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 tremor events during a 5 minute period. In embodiments, treatment reduces the number of tremor events during a 5 minute period compared to a control by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In embodiments, treatment reduces the number of tremor events during a 5 minute period compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, one or more of any value therein or range therebetween, or combinations thereof.

In embodiments, efficacy is determined by measuring average length of tremor events after treatment. In embodiments, after treatment, the tremor events have an average length of about 1, 2, 3, 4, 5, 6, 7, 8, or 9 seconds, or any value therein or range therebetween. In embodiments, treatment reduces the average length of tremor events compared to a control by about 1, 2, 3, 4, 5, 6, 7, 8, 9, or 10 or more seconds. In embodiments, treatment reduces the average length of tremor events compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, one or more of any value therein or range therebetween, or combinations thereof.

In embodiments, efficacy is determined by measuring how long a subject after treatment spends in tremor during a 5 minute period. In embodiments, the tremor after treatment results in a subject spending about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or less than 300 seconds in tremor during a 5 minute period. In embodiments, treatment reduces the time spent in tremor during a 5 minute period compared to a control by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 seconds. In embodiments, treatment reduces the time spent in tremor during a 5 minute period by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, one or more of any value therein or range therebetween, or combinations thereof.

In embodiments, efficacy is determined by measuring how long a subject after treatment spends in tremor during a length of time. In embodiments, treatment reduces how long a subject spends in tremor during a length of time by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% compared to a control.

In embodiments, efficacy is determined by scoring on TETRAS. In embodiments, the tremor after treatment corresponds to a TETRAS score of 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, or 63.5. In embodiments, treatment reduces the TETRAS score by 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, or 64 compared to a control.

### EXAMPLES

### Example 1. Treatment of Harmaline-Induced Essential Tremor in Rats with the Pharmaceutical Composition Disclosed Herein

### Materials

Harmaline & Propranolol: Harmaline and propranolol were obtained from Sigma.

Subjects: Naïve rats (114; male; Sprague Dawley; aged 6-9 weeks; weight 190-369 g) were obtained from Charles River UK and acclimated to the testing room in home cages (standard caging; housed in groups of 4) up to 7 days following arrival at the test facility, with free access to food (Certified Rodent CR 14% Protein Rodent Diet, LabDiet® 5CR4) and water on a 12 h/12 h light/dark cycle from arrival (lights on at 7:00 AM). All subjects were weighed at the beginning of the study and assigned to treatment groups.

The pharmaceutical composition disclosed herein (“NBX”): Comprising Δ-9-tetrahydrocannabinol (THC) and cannabidiol (CBD), wherein the THC and CBD are present at a weight ratio ranging from about 0.5:1.5 to 1.5:0.5 (e.g., 1.1:1).

Tremor Monitor™ (“TM”): A TM from San Diego Instruments was obtained to measure movement waveforms (i.e., amplitude (“A”) (dBV) as a function of frequency (“f”) (Hz)) (threshold 10 dBV).

### Methods

NBX/Harmaline Experimental Group (number of subjects (“n”)=36 (12 at each dose of NBX)): Subjects were habituated to the TM for 2×5 minutes (with a 20 minute gap between the two 5 minute periods). The next day, 5.2, 10.4, or 20.8 mg/kg (total THC and CBD) NBX was administered p.o. (oral gavage) in sesame oil to the subjects at 2 mL/kg. 100 minutes after the NBX was administered, 10 mg/kg of harmaline was administered i.p. in water to the subjects at 5 mL/kg. 20, 40, and 60 minutes after the harmaline was administered to the subjects, the subjects were observationally scored in their home cages on a scale from 0 to 4 (wherein scores (“observational scores” (“OS”)) of 0, 1, 2, 3, and 4 correspond, as summarized in TABLE A below, to “No tremor”, “Occasional tremor affecting only head and neck”, “Intermittent tremor affecting whole body”, “Persistent tremor affecting whole body and tail”, and “Severe tremor rendering subject unable to stand”, respectively) and, immediately thereafter, movement waveforms of the subjects were measured with the TM for 5 minutes each.

TABLE A
Observational scoring scale of tremors in subjects.

| Observational | |
|---|---|
| Scores (“OS”) | Corresponding Behavior |
| 0 | No tremor |
| 1 | Occasional tremor affecting only head and neck |
| 2 | Intermittent tremor affecting whole body |
| 3 | Persistent tremor affecting whole body and tail |
| 4 | Severe tremor rendering subject unable to stand |

Thereafter, the administration of the harmaline and NBX (“acute treatment”) was continued once per day until the harmaline and NBX had been administered for 10 days (“sub-chronic treatment”). 20, 40, and 60 minutes after the final administration of the NBX and harmaline, the subjects were again observationally scored on the scale described (and summarized in TABLE A) above and movement waveforms of the subjects were again measured with the TM for 5 minutes each. No adverse events (“AE”) were noted, even after sub-chronic treatment.

Propranolol/Harmaline Comparison Group (n=12): The Experimental Group protocol was repeated, except for administering, instead of NBX, 20 mg/kg of propranolol i.p. 30 minutes before the harmaline was administered (as the maximum effect of propranolol can be observed approximately 30 minutes after administration).

Propranolol/Vehicle Control Group (n=12): The Propranolol/Harmaline Comparison Group protocol was repeated, except for administering, instead of harmaline, saline (vehicle) at 5 mL/kg.

Vehicle/Vehicle Control Group (n=6): The Experimental Group protocol was repeated, except for administering, instead of NBX, sesame oil (vehicle) at 2 mL/kg, and, instead of harmaline, saline (vehicle) at 5 mL/kg.

Vehicle/Harmaline Control Group (n=12): The Experimental Group protocol was repeated, except for administering, instead of NBX, sesame oil (vehicle) at 2 mL/kg.

NBX/Vehicle Control Group (n=36 (12 at each dose)): The Experimental Group protocol was repeated, except for administering, instead of harmaline, saline (vehicle) at 5 mL/kg.

Calculations and Statistical Analysis: For each movement waveform measured, the following values were calculated: average amplitude (“A”) (average power) for the range of frequency (“f”) from

$$0 to 8.5 Hz ( “ AP 1 ” ( dBV ) = \frac{1}{( 8.5 - 0 )} {\sum }_{f = 0}^{8.5} {A}_{f} ) ;$$

average amplitude (“A”) (average power) for the range of frequency (“f”) from

$$9 to 12 Hz ( “ AP 2 ” ( dBV ) = \frac{1}{( 12 - 9 )} {\sum }_{f = 9}^{12} {A}_{f} ) ;$$

Tremor Index (“TI”=AP2−AP1); and Motion Power Percentage (“MPP” (%)=AP2/(AP1+AP2)×100). Further, for each subject, the following values were calculated: total number of tremor events (10 dBV threshold; sum of 20-, 40-, and 60-minute data); total average length of one tremor event (average of 20-, 40-, and 60-minute data); and total cumulative time of tremor events (sum of 20-, 40-, and 60-minute data), wherein “total” refers to combination of the 20-minute data, the 40-minute data, and the 60-minute data (e.g., “total cumulative time of tremor events” means total of the cumulative time of tremor events for the 20-minute data, the cumulative time of tremor events for the 40-minute data, and the cumulative time of tremor events for the 60-minute data). For the 20-, 40-, and 60-minute data independently, OS, MPP, and TI were analyzed by Kruskal-Wallis followed by Dunn's multiple comparison test. For the 20-, 40-, and 60-minuted data combined, the total number of tremor events, total average length (duration) of one tremor event, and total cumulative time spent in tremor were analyzed by Kruskal-Wallis followed by a Tukey's multiple comparison test. Results were considered significant for p<0.05. Outlier removal was not applied due to non-responders in the harmaline group which could not be identified in the treatment groups.

### Results

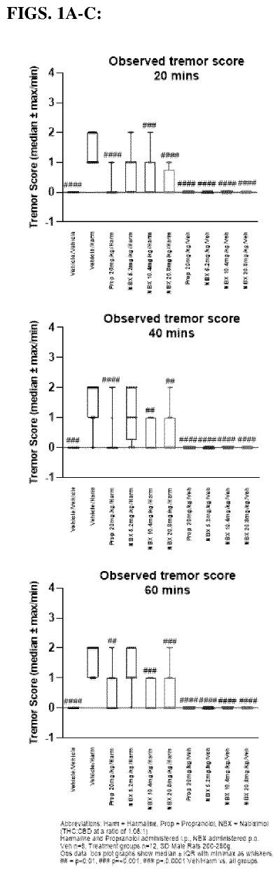

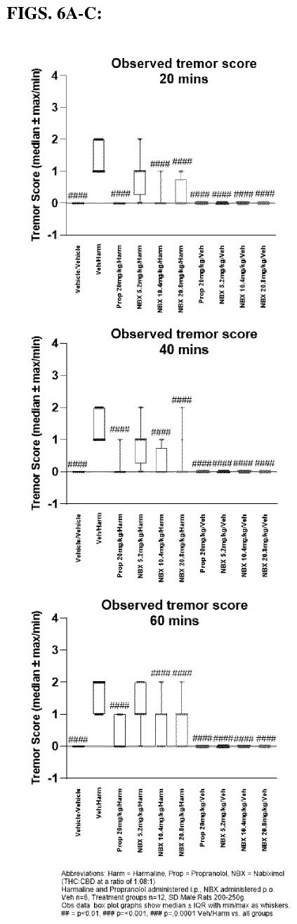

Tremor Score: For both acute treatment (FIGS. 1A-C) and sub-chronic treatment (FIGS. 6A-C): harmaline (10 mg/kg) significantly increased OS at 20, 40, and 60 minutes post-dose (“all timepoints”) (as shown by comparing the Vehicle/Harmaline Control Group to the Vehicle/Vehicle Control Group); each of propranolol (20 mg/kg) and, surprisingly, NBX (10.4 and 20.8 mg/kg total THC and CBD) significantly decreased OS at all time points (as shown by comparing the Propranolol/Harmaline Control Group and the NBX/Harmaline Experimental Group to the Vehicle/Harmaline Control Group); and the Propranolol/Vehicle Control Group and the NBX/Vehicle Control Group showed no significant effect compared to the Vehicle/Vehicle Control Group.

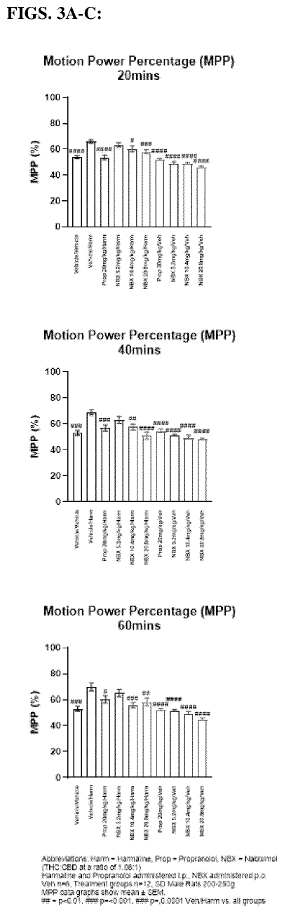

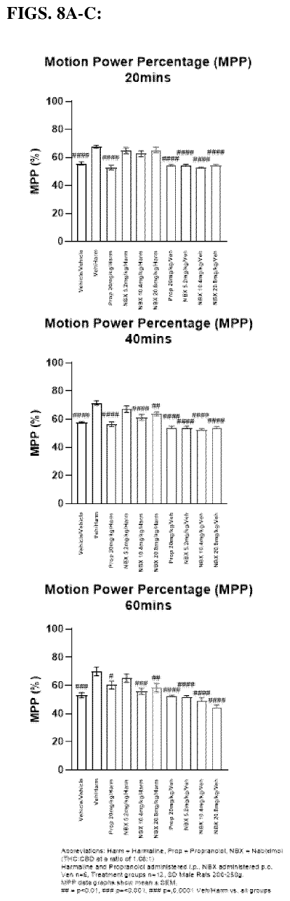

Motion Power Percentage (MPP): For both acute treatment (FIGS. 3A-C) and sub-chronic treatment (FIGS. 8A-C): harmaline (10 mg/kg) significantly increased MPP at all timepoints (as shown by comparing the Vehicle/Harmaline Control Group to the Vehicle/Vehicle Control Group); each of propranolol (20 mg/kg) and, surprisingly, NBX (10.4 and 20.8 mg/kg total THC and CBD) significantly decreased MPP at all timepoints (as shown by comparing the Propranolol/Harmaline Control Group and the NBX/Harmaline Experimental Group to the Vehicle/Harmaline Control Group); and, except for NBX at 20.8 mg/kg total THC and CBD at the 20 minute timepoint, the Propranolol/Vehicle Control Group and the NBX/Vehicle Control Group showed no significant effect compared to the Vehicle/Vehicle Control Group.

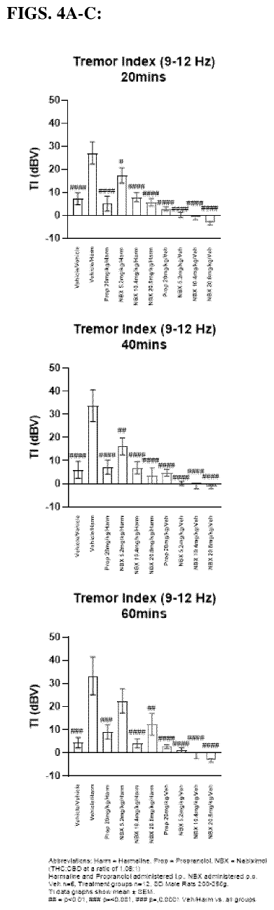

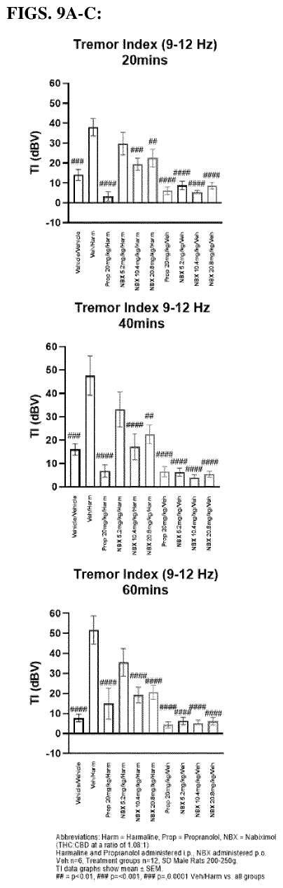

Tremor Index (TI): For both acute treatment (FIGS. 4A-C) and sub-chronic treatment (FIGS. 9A-C): harmaline (10 mg/kg) significantly increased TI at all timepoints (as shown by comparing the Vehicle/Harmaline Control Group to the Vehicle/Vehicle Control Group); each of propranolol (20 mg/kg) and, surprisingly, NBX (10.4 and 20.8 mg/kg total THC and CBD) significantly decreased TI at all timepoints (as shown by comparing the Propranolol/Harmaline Control Group and the NBX/Harmaline Experimental Group to the Vehicle/Harmaline Control Group); and the Propranolol/Vehicle Control Group and the NBX/Vehicle Control Group showed no significant effect compared to the Vehicle/Vehicle Control Group.

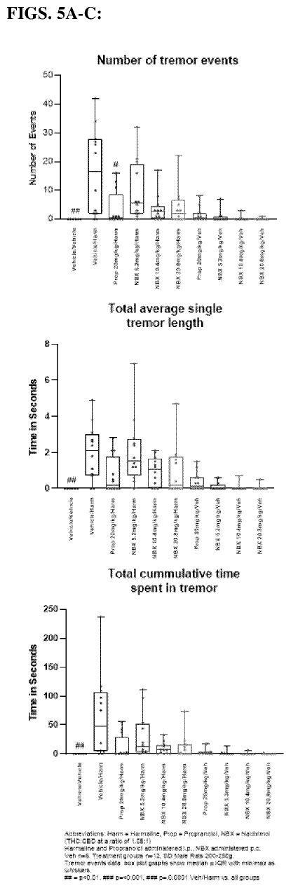

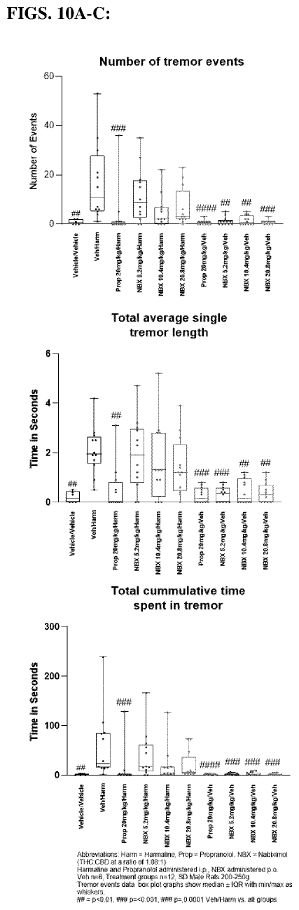

Total Number of Tremor Events, Total Average Length of One Tremor Event, and Total Cumulative Time of Tremor Events: For both acute treatment (FIGS. 5A-C) and sub-chronic treatment (FIGS. 10A-C): harmaline (10 mg/kg) significantly increased the total number of tremor events (FIG. 5A and FIG. 10A), total average length of one tremor event (FIG. 5B and FIG. 10B), and total cumulative time of tremor events (FIG. 5C and FIG. 10C) (as shown by comparing the Vehicle/Harmaline Control Group to the Vehicle/Vehicle Control Group). For acute treatment (FIGS. 5A-C), propranolol only significantly reduced the total number of tremor events (as shown by comparing the Propranolol/Harmaline Control Group to the Vehicle/Harmaline Control Group); while, for sub-chronic treatment (FIGS. 10A-C), propranolol significantly reduced the total number of tremor events, total average length of one tremor event, and total cumulative time of tremor events (as shown by comparing the Propranolol/Harmaline Control Group to the Vehicle/Harmaline Control Group).

### CONCLUSIONS

Overall, each of propranolol (20 mg/kg) and, surprisingly, NBX (10.4 and 20.8 mg/kg total THC and CBD) significantly reduced tremor, both acutely and sub-chronically, as indexed by OS, TI, and MPP. The minimum effective dose (MED) of NBX, both acutely and sub-chronically, was ≤10.4 mg/kg total THC and CBD. Further, surprisingly, no tachyphylaxis following sub-chronic treatment with NBX was noted.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### INCORPORATION BY REFERENCE

All publications, patents, and patent applications mentioned in this specification are herein incorporated by reference to the same extent as if each individual publication, patent, or patent application was specifically and individually indicated to be incorporated by reference. This specification will govern in the instance that there is any inconsistency between this specification and the publications, patents, and patent applications mentioned in this specification.

What is claimed is:

1. A method of treating a tremor in a subject, the method comprising administering to the subject a therapeutically effective amount of a pharmaceutical composition comprising Δ-9-tetrahydrocannabinol (THC) and cannabidiol (CBD), wherein the THC and CBD are present at a weight ratio ranging from about 0.5:1.5 to about 1.5:0.5.

2. The method of claim 1, wherein the subject is a human.

3. The method of claim 1, wherein the subject is a rat.

4. The method of claim 1, wherein the tremor comprises essential tremor.

5. The method of any one of claims 1-4, wherein the administration reduces an intensity of the tremor compared to a control.

6. The method of claim 5, wherein the administration reduces an amplitude of the tremor compared to a control.

7. The method of claim 6, wherein the administration reduces an integral of the amplitude of the tremor compared to a control.

8. The method of any one of claims 1-7, wherein about 0.001 to about 300 mg/kg/day THC and about 0.001 to about 300 mg/kg/day CBD are administered.

9. The method of any one of claims 1-8, wherein about 0.1 to about 100 mg/kg/day THC and about 0.1 to about 100 mg/kg/day CBD are administered.

10. The method of any one of claims 1-7, wherein about 0.1 to about 100 mg THC and about 0.1 to about 100 mg CBD are administered.

11. The method of any one of claims 1-10, wherein the THC and CBD are present in a weight ratio ranging from about 0.9:1.1 to about 1.1:0.9.

12. The method of any one of claims 1-11, wherein the THC and CBD are present in a weight ratio of about 1.1:0.9.

13. The method of any one of claims 1-12, wherein the pharmaceutical composition comprises a botanical drug substance comprising THC and CBD.

14. The method of any one of claims 1-13, wherein the pharmaceutical composition further comprises one or more cannabinoids in addition to THC and CBD.

15. The method of claim 14, wherein the one or more cannabinoids comprise cannabidiolic acid (CBDA), cannabidivarin (CBDV), cannabidiol-C1 (CBD-C1), cannabidiol-C4 (CBD-C4), tetrahydrocannabivarin (THCV), cannabigerol (CBG), hydroxy cannabidiol (OH-CBD), butyl-cannabidiol (CBD-C4), cannabicyclol (CBL), or a combination thereof.

16. The method of any one of claims 1-15, wherein the pharmaceutical composition further comprises one or more terpenes.

17. The method of any one of claims 1-16, wherein the pharmaceutical composition further comprises one or more sesquiterpenes.

18. The method of claim 16 or 17, wherein the one or more terpenes or sesquiterpenes comprise beta-farnesene, selina-3,7(11)-diene, guaia-3,9-diene, trans-caryophyllene, alpha-caryophyllene, trans-nerolidol, myrcene, trans-phytol, squalene, alpha-tocopherol, or a combination thereof.

19. The method of any one of claims 1-18, wherein the pharmaceutical composition further comprises one or more sterols.

20. The method of claim 19, wherein the one or more sterols comprise beta-sitosterol, beta-amyrin, campesterol, lupeol, or a combination thereof.

21. The method of any one of claim 1-20, wherein the pharmaceutical composition is administered orally.

22. The method of claim 21, wherein the pharmaceutical composition is a spray.

23. The method of claim 21, wherein the pharmaceutical composition is a tablet, a gel capsule, a mucoadhesive gel, a powder, a granule, or an extrudate.
